(19) United States
(12) Patent Application Publication
(10) Pub. No.: US 2003/0166707 A1
(43) Pub. Date: Sep. 4, 2003

(54) Microsphaeropsin B as novel antibacterial agent, process for producing the same and pharmaceutical composition containing thereof

(76) Inventors: Chao-Mei Yu, Ottawa (CA); Stephen W. Ayer, Vancouver (CA); Ronald G. Micetich, Sherwood Park (CA); Sameeh M. Salama, Sherwood Park (CA); Rakhshandeh Fathi-Afshar, Burnaby (CA); Jonathan M. Curtis, Halifax (CA); Jeffrey Lawson Cameron Wright, Wilmington, NC (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/272,309

(22) Filed: Oct. 17, 2002

Related U.S. Application Data
(60) Provisional application No. 60/329,537, filed on Oct. 17, 2001.

(51) Int. Cl. A61K 31/4015; C07D 207/12

(52) U.S. Cl. 514/424; 548/544

(57) ABSTRACT

One of the aspects of the present invention is directed to tetramic acid derivatives useful in treating or preventing bacterial diseases, especially diseases caused by gram-positive pathogens resistant to antibiotics of the prior art. Within the scope of the present invention are pharmaceutical compositions containing at least one of the tetramic acid derivatives of the invention as the active ingredient, methods of treating and/or preventing a bacterial disease by administering at least one of the tetramic acid derivatives of the invention, and the use of the tetramic acid derivatives of the invention in the treatment and/or prevention of a bacterial disease. Preferably, the tetramic acid derivative of the invention is a compound of formula IV shown below.

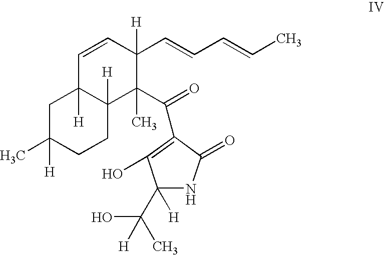

This application claims the benefit of U.S. Provisional Patent Application, No. 60/329,537, filed Oct. 17, 2001, the disclosure of which is incorporated by reference.

### BACKGROUND OF THE INVENTION

The present invention relates to tetramic acid derivatives, in particular Microsphaeropsin B, as antibiotics having antibacterial activities, especially against Gram positive pathogens including Vancomycin resistant Enterococcus faecalis (VRE), a process for the production thereof and a pharmaceutical composition containing at least one of the tetramic acid derivatives.

Bacteria are very adaptable microorganisms that possess the ability to adapt and to survive under adverse conditions. Doctors in hospitals and clinics around the world are losing the battle against an onslaught of new drug resistant bacterial infections including those caused by Staphylococci, Streptococci, Enterococci and Pseudomonas.

Bacterial resistance to the current antibiotics has been on a steep rise due to target alterations, a change in permeability pattern, efflux of active ingredients and/or deactivation of the antibiotics before reaching the active sites.

Tetramic acids (2,4-pyrrolidinediones) are an increasingly growing structural class of agents, both natural and synthetic, many members of which possess interesting and varied biological properties including antimicrobial, antitumor and cytotoxic activities. A number of microbial metabolites having a tetramic acid pharmacophore are known in the literature, such as “Equistein” (U.S. Pat. No. 3,959,468), “Vermisporin” (U.S. Pat. No. 4,933,180) and “Lydicamycin” (Journal of Antibiotics, 44:282-292, 1991) as antibiotics and “Cryptocin” (Organic Letters 2:767-770, 2000) as an antifungal agent.

There is a need of antibacterial agents for treating diseases caused by bacteria resistant to currently available antibiotics. The present invention addresses that need by providing tetramic acid derivatives having anti-bacterial activities, especially useful in treating diseases caused by antibiotic-resistant bacteria.

### SUMMARY OF THE INVENTION

One of the objects of the present invention is directed to tetramic acid derivatives, which are Microsphaeropsin B produced from the cultivation of a strain of fungus belonging to the genus Microsphaeropsis and the derivatives of Microsphaeropsin B. The derivatives of Microsphaeropsin B include dehydromicrosphaeropsin B, hexahydromicrosphaeropsin B, and compounds prepared from derivatization of at least one of the hydroxyl and/or carbonyl groups of Microsphaeropsin B, dehydromicrosphaeropsin B or hexahydromicrosphaeropsin B. The tetramic acid derivatives, especially Microsphaeropsin B, of the present invention are useful for treating or preventing diseases caused by bacteria, especially gram-positive pathogens including vancomycin resistant Entrococcus faecalis (VRE). The physicochemical and antibacterial properties of Microsphaeropsin B are described below.

The present invention is also directed to a pharmaceutical composition containing at least one of the tetramic acid derivatives of the invention for the treatment or prevention of diseases caused by bacteria, preferably gram-positive pathogens, and more preferably gram-positive pathogens, such as VRE, resistant to currently available antibiotics. Another object of the present invention is directed to processes for preparing the tetramic acid derivatives of the invention, especially a process for the preparation of Microsphaeropsin B.

The present invention relates to tetramic acid derivatives of formula I,

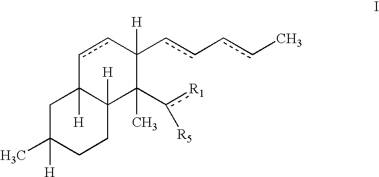

wherein

R₁ is O or OH (preferably, R₁ is O);

R₅ is a group represented by formula II or III (preferably, R₅ is a group of formula II),

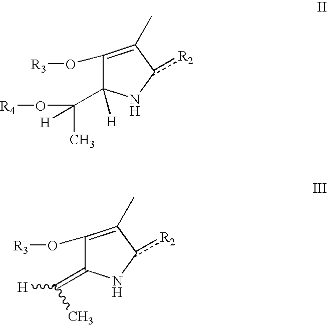

R₂ is O or OH (preferably, R₂ is O);

each of the dashed lines independently represents a single bond or nothing so that the two atoms attached to the two ends of the dashed line are connected by a single or double bond, wherein the dashed line attached to R₁ represents a single bond when R₁ is O or nothing when R₁ is OH, and the dashed line attached to R₂ represents a single bond when R₂ is O or nothing when R₂ is OH;

R₃ is H, C₁-C₆ alkyl, —C(O)R₁₁, —C(O)R₁₂, —P(O)(OH)₂, an amino acid residue, a dicarboxylic acid residue, —C(O)—C(O)R₁₃ or —C(O)OR₁₄ (preferably, R₃ is H);

R₄ is H, C₁-C₆ alkyl, —C(O)R₄₁, —C(O)R₄₂, —P(O)(OH)₂, an amino acid residue, a dicarboxylic acid residue, —C(O)—C(O)R₄₃ or —C(O)OR₄₄ (preferably, R₄ is H);

the amino acid residue is an amino acid having OH removed from a carboxyl group;

the dicarboxylic acid residue is a dicarboxylic acid having OH removed from one of the carboxyl groups;

R₁₁ and R₄₁ are each independently (C₁-C₂₀)alkyl, (C₁-C₂₀)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₂₀)alkenyl, (C₂-C₂₀)alkenyl substituted by one to three halogen atoms, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;

R₁₂ and R₄₂ are each independently —N(R₂₀)(R₂₁), 1-imidazolyl or pyrrolyl;

R₂₀ and R₂₁ are each independently H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;

R₁₃ and R₄₃ are each independently (C₁-C₆)alkyl or (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups; and

R₁₄ and R₄₄ are each independently (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;

wherein the heterocyclic group is a 5- or 6-membered ring containing one to three N, O and/or S ring atoms optionally fused with a benzene or 5- or 6-membered ring containing one to three N, O and/or S ring atoms; or

a pharmaceutically acceptable salt, solvate or hydrate thereof.

A preferred embodiment of the compounds of formula I, is Microsphaeropsin B represented by formula IV,

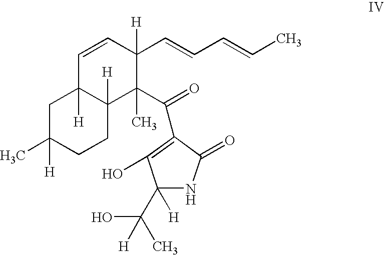

or a pharmaceutically acceptable salt, solvate or hydrate thereof.

The scope of the present invention includes (a) substantially purified or isolated Microsphaeropsin B, or (b) optionally purified or isolated, to a substantial degree, compounds of formula I other than Microsphaeropsin B.

Also within the scope of the present invention are pharmaceutically acceptable salts, solvates or hydrates of the tetramic acid derivatives of the present invention.

### BRIEF DESCRIPTION OF THE DRAWINGS

FIG. 1 is the ¹H NMR spectrum of Microsphaeropsin B.

FIG. 2 is the ¹³C NMR spectrum of Microsphaeropsin B.

### DETAILED DESCRIPTION OF THE INVENTION

In this application, the term “heterocyclic group” represents a 5- or 6-membered ring containing one to three N, O and/or S ring atoms optionally fused with a benzene or 5- or 6-membered ring containing one to three N, O and/or S ring atoms. Examples of “heterocyclic group” include piperinyl, piperazinyl, morpholinyl, thiomorpholinyl, indolinyl, isoindolinyl, thienyl, furanyl, pyrrolyl, pyrrolidinyl, imidazolyl, oxazolyl, thiazolyl, isooxazolyl, isothiazolyl, thiadazolyl, 1H-pyrazolyl, pyridinyl, pyridazinyl, pyrimidinyl, pyrazinyl, benzopyranyl, benzofuranyl, benzo[b]thiophenyl, indolyl, isoindolyl, indolizinyl, benzimidazolyl, benz[d]isoxazolyl, benzotriazolyl, chromanyl, indazolyl, purinyl, quinolinyl, isoquinolinyl, quinolizinyl, quinazolinyl, cinnolinyl, quinazolinyl, quinoxalinyl, phthalazinyl, pteridinyl and naphthyridinyl.

The term “heteroaryl”, used herein, refers to an aromatic 5- or 6-membered ring containing one to three N, S and/or O ring atoms optionally fused with a benzene or aromatic 5- or 6-membered ring containing one to three N, S and/or O ring atoms. Examples of “heteroaryl” include thienyl, furanyl, pyrrolyl, imidazolyl, oxazolyl, thiazolyl, isooxazolyl, isothiazolyl, thiadazolyl, pyridinyl, pyridazinyl, pyrimidinyl, pyrazinyl, benzofuranyl, benzo[b]thiophenyl, indolyl, isoindolyl, benzimidazolyl, benz[d]isoxazolyl, benzotriazolyl, indazolyl, purinyl, quinolinyl, isoquinolinyl, cinnolinyl, quinazolinyl, quinoxalinyl, phthalazinyl, naphthyridinyl and pteridinyl.

The term “halogen” means fluorine, chlorine, bromine or iodine, with chlorine and bromine being preferred. The term “halogen groups” represent halogen atoms having a valence of one, and examples are fluoro, chloro, bromo and iodo radicals, wherein “halogen groups” preferably are chloro and bromo radicals. The term “halogen”, in combination with another term, represents the modification of the other term with one or more fluorine, chlorine, bromine or iodine atoms, with chlorine and bromine being preferred. For example, “trihalogenated phenyl” means a phenyl group substituted with three halogen groups.

“Alkyl”, used herein, refers to a straight-chain or branched saturated hydrocarbyl, preferably of 1 to 20 carbon atoms. Examples of “alkyl” or “(C₁-C₂₀)alkyl” include methyl, ethyl, n-propyl, isopropyl, n-butyl, sec-butyl, isobutyl, tert-butyl, n-pentyl, sec-pentyl, tert-pentyl, 2-methylbutyl, 3-methylbutyl, 2,2-dimethylpropyl, n-hexyl, 1-methylpentyl, 2-methylpentyl, 3-methylpentyl, isohexyl, 4,4-dimethylpentyl, n-heptyl, isoheptyl, n-octyl, iso-octyl, n-nonyl, isononyl, n-decyl, n-undecyl, 4-ethyl-3,3-dimethylheptyl, n-dodecyl, n-tridecyl, n-tetradecyl, n-pentadecyl, n-hexadecyl, n-heptadecyl, n-octadecyl, n-nonadecyl and n-icosyl. The preferred examples of “alkyl” or “(C₁-C₂₀)alkyl” are methyl, ethyl, n-propyl, isopropyl, n-undecyl, n-tridecyl, n-pentadecyl, n-heptadecyl and n-nonadecyl. Examples of “(C₁-C₆)alkyl” are methyl, ethyl, n-propyl, isopropyl, n-butyl, sec-butyl, isobutyl, tert-butyl, n-pentyl, sec-pentyl, tert-pentyl, 2-methylbutyl, 3-methylbutyl, 2,2-dimethylpropyl, n-hexyl, 1-methylpentyl, 2-methylpentyl, 3-methylpentyl, 3,3-dimethylbutyl and isohexyl. “(C₁-C₆)alkyl” preferably is methyl, ethyl, n-propyl, isopropyl, n-butyl, sec-butyl, isobutyl or tert-butyl, more preferably methyl or ethyl, and most preferably methyl. The term, “alkyl”, used in combination with another term represents a modification of the other term by one or more alkyl groups as defined above. Similarly, the term, “(C₁-C₆)alkyl”, used in combination with another term represents a modification of the other term by at least one (C₁-C₆)alkyl group as defined above. For instance, “mono-(C₁-C₆)alkyl-amino” means an amino group substituted by one (C₁-C₆)alkyl group; and “dialkylated phenyl” means a phenyl group substituted by two alkyl groups.

The term “(C₂-C₂₀)alkenyl” represents a straight-chain or branched unsaturated hydrocarbyl radical of 2 to 20 carbon atoms. Examples of “(C₂-C₂₀)alkenyl” are ethenyl, allenyl, 1-propenyl, allyl, butenyl, 1-methylvinyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl and icosenyl. Preferred examples of “(C₂-C₂₀)alkenyl” are ethenyl, 1-propenyl and allyl. Undecenyl, tridecenyl, pentadecenyl, heptadecenyl and nonadecenyl are also preferred examples of “(C₂-C₂₀)alkenyl”. Examples of “(C₂-C₂₀)alkenyl” more preferably are undecenyl, tridecenyl, pentadecenyl, heptadecenyl and nonadecenyl, and most preferably are 8-pentadecenyl, 8-heptadecenyl, 8,11-heptadecadienyl, 8,11,14-heptadecatrienyl, 5,8,11-heptadecatrienyl, 4,7,10,13-nonadecatetraenyl, and 4,7,10,13,16-nonadecapentaenyl.

In this application, “(C₂-C₆)alkenyl” represents a straight-chain or branched unsaturated hydrocarbyl radical of 2 to 6 carbon atoms. Examples of “(C₂-C₆)alkenyl” are ethenyl, allenyl, 1-propenyl, allyl, butenyl, 1-methylvinyl, pentenyl and hexenyl. Preferred examples of “(C₂-C₆)alkenyl” are ethenyl, allenyl, 1-propenyl, allyl and butenyl. Most preferably, “(C₂-C₆)alkenyl” are ethenyl, 1-propenyl and allyl.

The term, “(C₄-C₈)cycloalkyl”, means a cyclic saturated hydrocarbyl having 4, 5, 6, 7 or 8 carbon ring atoms. Examples of “(C₄-C₈)cycloalkyl” include cyclobutyl, cyclopentyl, cyclohexyl, cycloheptyl and cyclooctyl. Preferably, “(C₄-C₈)cycloalkyl” is cyclopentyl, cyclohexyl or cycloheptyl. “(C₄-C₈)cycloalkyl”, more preferably, is cyclopentyl or cyclohexyl.

In the compounds of formula I according to the invention, R₅ is either a group of formula II or III. Preferably, R₅ is a group of formula II.

In this application, “amino acid residue” means a radical with the removal of a hydroxyl group from an amino acid. In some of the embodiments of the compounds of formula I according to the present invention, R₃ and/or R₄ is an amino acid residue. Preferably, the amino acid residue is —C(O)—C(R₂₄)(R₂₅)—N(R₂₂)(R₂₃), —C(O)—C(R₃₀)(R₃₁)—C(R₂₈)(R₂₉)—N(R₂₆)(R₂₇), —C(O)—C(R₃₈)(R₃₉)—C(R₃₆)(R₃₇)—C(R₃₄)(R₃₅)—N(R₃₂)(R₃₃), 2-pyrrolidinylcarbonyl or 4-hydroxy-2-pyrrolidinylcarbonyl; wherein

R₂₂, R₂₃, R₂₅, R₂₆, R₂₇, R₂₉, R₃₁, R₃₂, R₃₃, R₃₅, R₃₇ and R₃₉ are each independently H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a C₄-C₈ cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;

R₂₄ is H, unsubstituted (C₁-C₆)alkyl or (C₁-C₆)alkyl substituted by one to three substituents selected from thio, OH, (C₁-C₆)alkylthio, (C₁-C₆)alkoxy, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;

one of R₂₈ and R₃₀ is H, (C₁-C₆)alkyl or (C₁-C₆)alkyl substituted by one to three substituents selected from thio, OH, (C₁-C₆)alkylthio, (C₁-C₆)alkoxy, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;

the remaining of R₂₈ and R₃₀ is H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, C₁-C₆ alkyl or C₂-C₆ alkenyl groups, or a cyclic moiety which is a (C₄-C₈)cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;

one of R₃₄, R₃₆ and R₃₈ is H, unsubstituted (C₁-C₆)alkyl or (C₁-C₆)alkyl substituted by one to three substituents selected from thio, OH, (C₁-C₆)alkylthio, (C₁-C₆)alkoxy, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;

the remaining of R₃₄, R₃₆ and R₃₈ are H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups.

In the compounds of formula I comprising the amino acid residue according to the invention, more preferably,

R₂₄ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl;

one of R₂₈ and R₃₀ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, CH₂—OP(O)(OH)₂, 2,2-dicarboxylethyl, 4-(acetylamino)butyl or 3-aminopropyl; and

one of R₃₄, R₃₆ and R₃₈ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl.

Within the scope of the invention are compounds of formula I, wherein R₃ and R₄ are each independently H, methyl, ethyl or the amino acid residue; and at least one of R₃ and R₄ is the amino acid residue. One of the objects of the invention is directed to compounds of formula I, wherein one of R₃ and R₄ is the amino acid residue; the remaining one of R₃ and R₄ is H; and R₁ and R₂ are O. In some of the compounds of formula I, preferably, R₃ is the amino acid residue and R₄ is H; or R₄ is the amino acid residue and R₃ is H.

In some of the embodiments of the compounds of formula I according to the invention, R₃ and/or R₄ is a dicarboxylic acid residue. The term, “dicarboxylic acid residue”, represents a radical formed by removing a hydroxy group from one of the carboxyl groups of a dicarboxylic acid.

Preferably, the “dicarboxylic acid residue” is —C(O)—R₄₀—C(O)OH, wherein R₄₀ is a divalent (C₁-C₂₀)hydrocarbyl group, which is unsubstituted or substituted by one to three halogen atoms, OH, SH, nitro, amino, mono-(C₁-C₆)alkyl-amino, di-(C₁-C₆)alkyl-amino or (C₁-C₆)alkyl-carbonyl groups. R₄₀ preferably is (C₁-C₆)alkylene, (C₁-C₆)alkylene substituted by one to three halogen atoms, OH, SH, nitro, amino, mono-(C₁-C₆)alkyl-amino, di-(C₁-C₆)alkyl-amino or (C₁-C₆)alkyl-carbonyl groups, (C₂-C₆)alkenylene, (C₂-C₆)alkenylene substituted by one to three halogen atoms, OH, SH, nitro, amino, mono-(C₁-C₆)alkyl-amino, di-(C₁-C₆)alkyl-amino or (C₁-C₆)alkyl-carbonyl groups, a bivalent cyclic moiety which is (C₄-C₈)cycloalkylene, phenylene or naphthylene, —CH₂-R₄₁— or —R₄₁-CH₂—, wherein the cyclic moiety is unsubstituted or substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups; and R₄₁ is 1,2-phenylene, 1,2-phenylene substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, 1,3-phenylene, 1,3-phenylene substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, 1,4-phenylene, or 1,4-phenylene substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups.

More preferably, R₄₀ is —(CH₂)n—, —CH═CH—, —C(CH₃)═CH—, —CH═C(CH₃)—, —C(CH₃)═C(CH₃)—, —CH═CHCH₂—, —C(CH₃)═CHCH₂—, —CH═C(CH₃)CH₂—, —C(CH₃)═C(CH₃)CH₂—, —CH₂CH═CH—, —CH₂C(CH₃)═CH—, —CH₂CH═C(CH₃)—, —CH₂C(CH₃)═C(CH₃)—, —CH₂CH═CHCH₂—, —CH₂C(CH₃)═CHCH₂—, —CH₂CH═C(CH3)CH2—, —CH₂C(CH₃)═C(CH3)CH2—, —C(CH₃)CH═CHCH₂—, —CH₂CH═CHCH(CH₃)—, —CH(CH₃)CH═CHCH(CH₃)—, 1,2-phenylene, 1,3-phenylene or 1,4-phenylene; and n is an integer of 1, 2, 3 or 4 (preferably, n is 2 or 3).

In some of the embodiments of the compounds of formula I, R₃ and R₄ are each independently H, methyl, ethyl or the dicarboxylic acid residue; at least one of R₃ and R₄ is the dicarboxylic acid residue (preferably, R₁ and R₂ are O). In these embodiments, preferably, one of R₃ and R₄ is the dicarboxylic acid residue; the remaining one of R₃ and R₄ is H or methyl. More preferably, R₃ is the dicarboxylic acid residue and R₄ is H; or R₄ is the dicarboxylic acid residue and R₃ is H.

The present invention also provides the compounds of formula I, wherein R₃ is H, methyl, ethyl or —C(O)—R₁₂, R₄ is H, methyl, ethyl or —C(O)—R₄₂ with at least one of R₃ and R₄ being —C(O)—R₁₂ or —C(O)—R₄₂ (preferably, R₁ and R₂ are O). Preferably, in these compounds of formula I, R₃ is —C(O)—R₁₂ or R₄ is —C(O)—R₄₂. More preferably, R₃ is —C(O)—R₁₂ and R₄ is H, or R₄ is —C(O)—R₄₂ and R₃ is H. R₁₂ and R₄₂ are each independently —N(R₂₀)(R₂₁), 1-imidazolyl or pyrrolyl, wherein R₂₀ and R₂₁ are as defined above. R₁₂ and R₄₂, preferably, are each independently —N(R₂₀)(R₂₁), 1-imidazolyl or pyrrolyl, wherein R₂₀ and R₂₁ are independently H or (C₁-C₆)alkyl; and most preferably, one of R₁₂ and R₄₂ is —N(R₂₀)(R₂₁).

Another object of the invention is directed to the compounds of formula I, wherein R₃ is H, methyl, ethyl or —C(O)—R₁₁ and R₄ is H, methyl, ethyl or —C(O)—R₄, with at least one of R₃ and R₄ being —C(O)—R₁₁ or —C(O)—R₄₁ (preferably, R₁ and R₂ are O). In these compounds of formula I, preferably, R₃ is —C(O)—R₁₁ or R₄ is —C(O)—R₄₁. More preferably, R₃ is —C(O)—R₁₁ and R₄ is H, or R₄ is —C(O)—R₄₁ and R₃ is H.

Within the scope of the invention are the compounds of formula I, wherein R₃ and R₄ are each independently H, methyl, ethyl or —P(O)(OH)₂ with at least one of R₃ and R₄ being —P(O)(OH)₂ (preferably, R₁ and R₂ are O). In these compounds of formula I, preferably, one of R₃ and R₄ is —P(O)(OH)₂. More preferably, in these compounds, R₃ is —P(O)(OH)₂ or R₄ is H, or R₄ is —P(O)(OH)₂ or R₃ is H.

The present invention also provides the compounds of formula I, wherein R₃ is H, methyl, ethyl or —C(O)—C(O)—R₁₃ and R₄ is H, methyl, ethyl or —C(O)—C(O)—R₄₃ with at least one of R₃ and R₄ being —C(O)—C(O)—R₁₃ or —C(O)—C(O)—R₄₃ (preferably, R₁ and R₂ are O). Preferably, in these compounds of formula I, R₃ is —C(O)—C(O)—R₁₃ and R₄ is H, or R₃ is H and R₄ is —C(O)—C(O)—R₄₃, wherein R₁₃ preferably is (C₁-C₆)alkyl, more preferably R₁₃ is methyl or ethyl and most preferably R₁₃ is methyl.

One of the objects of the invention is directed to the compound of formula I, wherein R₃ is H, methyl, ethyl or —C(O)—O—R₁₄ and R₄ is H, methyl, ethyl or —C(O)—O—R₄₄ with at least one of R₃ and R₄ being —C(O)—O—R₁₄ or —C(O)—O—R₄₄ (preferably, R₁ and R₂ are O). Preferably, in these compounds of formula I, R₃ is —C(O)—C(O)—R₁₄ and R₄ is H, or R₃ is H and R₄ is —C(O)—C(O)—R₄₄, wherein R₁₄ is preferably (C₁-C₆)alkyl, more preferably methyl, ethyl, n-propyl, isopropyl, n-butyl, sec-butyl, isobutyl or tert-butyl, even more preferably methyl or ethyl, and most preferably methyl.

The tetramic acid derivatives represented by formula I of the present invention (preferably Microsphaeropsin B) have antibacterial activities, especially against gram-positive pathogens including vancomycin resistant Enterococcus faecalis (VRE). Microsphaeropsin B is represented by formula IV,

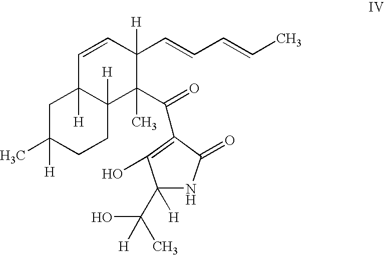

The compounds of formula I can be used to treat infections caused by bacteria, especially gram-positive bacteria including vancomycin resistant Enterococcus faecalis (VRE), alone or in combination with other drugs in mammals including humans.

It is an additional object of the invention to provide a method for the treatment or prevention of a disease caused by bacteria, especially gram-positive pathogenic bacteria including vancomycin resistant Entrococcus faecalis (VRE), in a subject in need of the treatment or prevention, by administering an antibacterial effective amount of a compound of formula I, in particular Microsphaeropsin B, to the subject. The subject is an animal, such as a human or another mammal; preferably, the subject is a human.

Within the scope of the present invention is a process for preparing the compounds of formula I. Preferably, the process is for preparing a substantially purified compound of formula IV, wherein the process includes the steps of cultivating a Microsphaeropsin producing strain of Microsphaeropsis olivacea in a growth promoting medium under aerobic condition at a temperature ranging from 20-30° C. to produce Microsphaeropsin B in the growth medium; and thereafter isolating Microsphaeropsin B from the growth medium to obtain a substantially pure compound of formula IV.

Microsphaeropsin B was isolated from the strain of fungus genus Microsphaeropsis according to the present invention and has following characteristics:

a. Elemental Composition:

| | |
|---|---|
| Carbon | 73.12% |
| Hydrogen | 8.32% |
| Nitrogen | 3.45%; |

b. Molecular Formula: C₂₄H₃₃NO₄;

c. Molecular Weight: 399.24;

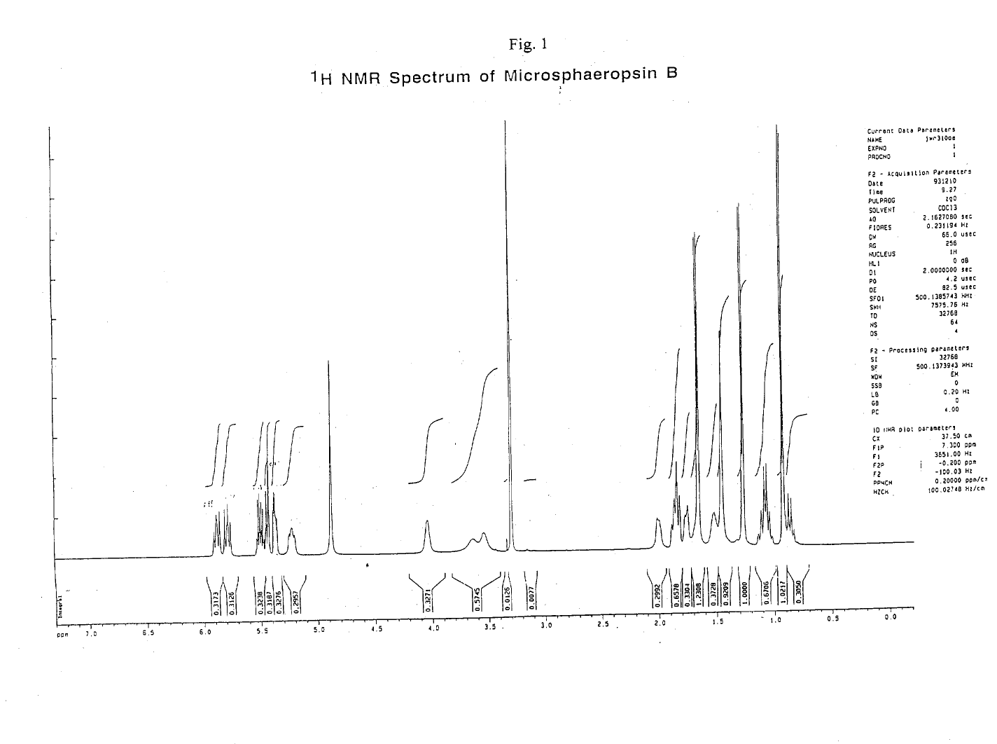

d. Proton NMR spectrum determined in CD₃OD solution as shown in FIG. 1;

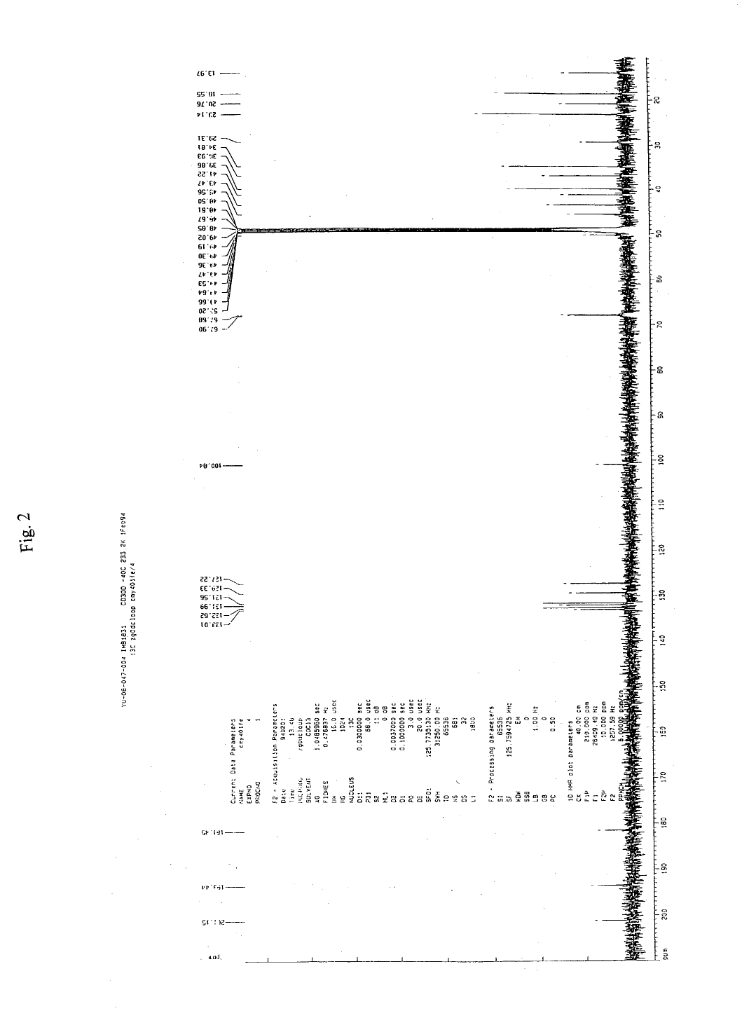

e. ¹³C-NMR spectrum determined in CD₃OD solution as shown in FIG. 2;

f. R_{f} value of 0.5 when subjected to reverse phase C₁₈ thin layer chromatography plate (5 cm height) using 5% H₂O in acetonitrile as developing solvent;

g. A dark color spot developed after heating with anisaldehyde; and

h. Solubility in methanol and insoluble in water, 5% sodium bicarbonate or 1M sodium hydroxide solution; and dissolution in ethanol or dimethyl sulphoxide followed by addition of water gave immediate precipitate formation.

The fungus, which produces Microsphaeropsin, was isolated from two different sponge samples collected in Florida. After collection, the sponge samples were immediately frozen over dry ice and transported to a laboratory for processing within a few days in a frozen state. Two strains of fungus were isolated: SF-10 was isolated from a sponge Angelus species A and SF-26 was isolated from a sponge Halichondria melandocia.

Both strains were isolated from the respective sponge samples by transferring small pieces of thawed sponge samples to separate sterile agar plates and allowing the fungal colonies to grow out. Seven days after adding the sponge sample, a sterile microbiological loop was used to transfer mycelia from SF-10 to a separate agar isolation plate. After confirmation that the culture of strain SF-10 was not contaminated with any other microorganisms, a sterile loop was used to transfer fungal mycelia to an agar slant for storage. The same procedure was used to isolate strain SF-26. Both the fungal strains SF-10 and SF-26 were identified as Microsphaeropsis olivacea.

For the isolation of novel compounds, both strains of M. olivacea were cultured in a liquid medium at a one-liter shake-flask scale (ten 250 mL flasks each containing 100 mL of the medium). A liquid culture inoculum for each strain was prepared by sterilely transferring mycelia from the agar storage slant to a 250 mL flask containing 100 mL of the liquid medium. After two days of shaking at 250 rpm, 5 mL aliquots of the inoculum culture were transferred to each of the ten production flasks. The production cultures were grown for seven days, with shaking at 250 rpm, and then harvested by filtration. The culture filtrate of SF-10 was extracted with butanol. The butanol extract was then evaporated to dryness to give a crude material. The culture extract of SF-26 was extracted with ethyl acetate and then the extract was evaporated to dryness to give a crude material.

To obtain greater quantities of the crude extracts for isolation of Microsphaeropsin B and its magnesium salt form, both strains were cultured at a six-liter shake-flask scale (sixty 250 mL flasks each containing 100 mL of the liquid medium). For the subsequent cultures of strain SF-10, ethyl acetate was generally used as the extraction solvent instead of butanol.

Microsphaeropsin can be isolated in either its magnesium salt form, labeled as Microsphaeropsin A, or as the free tetramic acid, labeled as Microsphaeropsin B. The magnesium salt form was obtained from an ethyl acetate extract of strain SF-26 using silica gel chromatography with final purification accomplished using Sephadex LH-20 chromatography. The free tetramic acid was isolated from an ethyl acetate extract of strain SF-10 using reversed-phase flash chromatography with final purification achieved using reversed-phase HPLC.

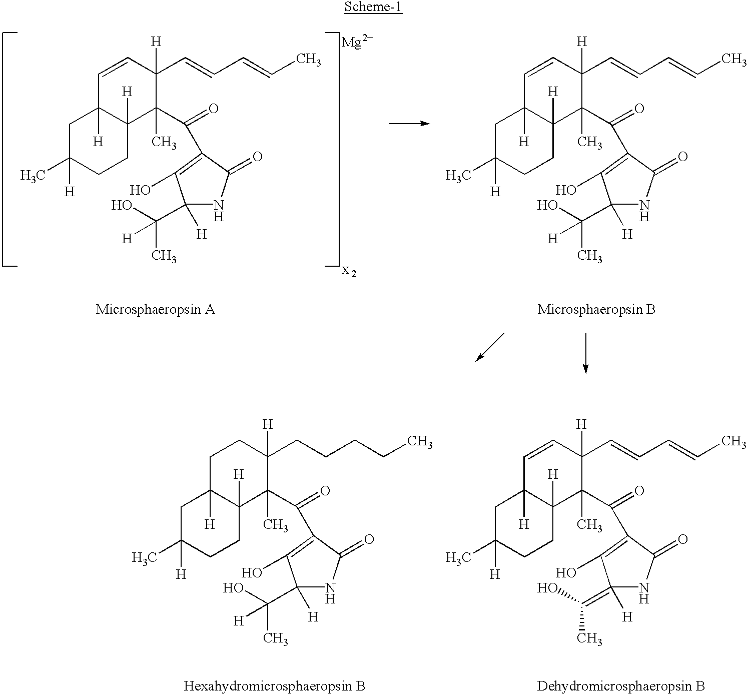

The structure of Microsphaeropsin B and its magnesium salt form were determined by Elemental analysis, exact mass measurement, proton NMR, ¹³C NMR and a number of NMR experiment such as DEPT, HMQC, 2-D COSY, HMBC and TOCSY. The proton and ¹³CNMR spectra of Microsphaeropsin B are reported in FIG. 1 and 2, respectively. The chemical structure of Microsphaeropsin B was confirmed by conversion of Microsphaeropsin to hexahydromicrosphaeropsin by hydrogenation with hydrogen at a pressure above atmospheric pressure or to dehydromicrosphaeropsin by hydrogenation with hydrogen at atmospheric pressure using a Pd/C catalyst as shown in scheme-1.

The proton NMR spectrum of the magnesium salt form of Microsphaeropsin B was complicated by the presence of minor resonances attributed to the presence of more than one distinct magnesium complex. The cation of the magnesium salt form of Microsphaeropsin B was determined by X-ray fluorescence spectroscopy and partial conversion of Microsphaeropsin B using magnesium sulfate. The magnesium salt form of Microsphaeropsin B can be converted to Microsphaeropsin B by passage through a cation exchange column equilibrated in the proton form.

The compounds of formula I, other than the compound of formula IV which can be prepared synthetically, can be prepared from Microsphaeropsin B using known procedures of organic chemical synthesis, such as disclosed in Organic Synthesis, Michael B. Smith, 2001, McGraw-Hill. Dehydromicrosphaeropsin can be prepared by hydrogenation of Microsphaeropsin B under atmospheric pressure in the presence of a Pd/C catalyst or isolated from the growth medium of SF-10 or SF-26 followed by chromatography. Hexahydromicrosphaeropsin can be obtained by hydrogenation of Microsphaeropsin B with hydrogen under a high pressure (above atmospheric) in the presence of a Pd/C catalyst. Other compounds of formula I can be obtained from Microsphaeropsin B, dehydromicrosphaeropsin B or hexahydromicrosphaeropsin by derivatization of one or both hydroxyl groups and/or reduction of one or both carbonyl groups when R₁ and R₂ are O according to procedures known in the art, e.g. see Advanced Organic Chemistry, Reactions, Mechanisms and Structure, Jerry March, 1992, Wiley-Interscience and Comprehensive Organic Transformations, Richard C. Larock, 1999, Wiley-VCH, after protection of some of the functional groups if necessary as discussed in Protective Groups in Organic Synthesis, T. W. Green and P. G. M. Wuts, 1999, John Wiley & Sons. For instance, the compounds of formula I wherein R₃ and/or R₄ is —C(O)—R₁₂ can be prepared by reacting Microsphaeropsin B, dehydromicrosphaeropsin B or hexahydromicrosphaeropsin with an activated form, such as the anhydride or acid chloride, of HOC(O)—R₁₂. The compounds of formula I wherein R₃ and/or R₄ is a dicarboxylic acid residue can be obtained by reacting Microsphaeropsin B, dehydromicrosphaeropsin B or hexahydromicrosphaeropsin with the anhydride form of the dicarboxylic acid, e.g. using a procedure similar to the one disclosed in U.S. Pat. No. 5,654,446.

It is a further object of the invention to provide a pharmaceutical composition suitable for the treatment or prevention of a bacterial disease, wherein the composition comprises the compound of formula I with a pharmaceutically acceptable carrier, diluent or excipient. The pharmaceutically acceptable carrier, diluent or excipient can be aqueous, which contains sterile water.

The pharmaceutical dosage forms include parenteral preparations such as injection formulations, suppositories, aerosols, creams and the like, and oral preparations such as uncoated tablets, coated tablets, powders, granules, capsules, liquids and the like. The above preparations are formulated in manners well known in the art.

For the formulation of injections, a pH adjusting agent, buffer, stabilizer, isotonic agent, local anesthetic or the like is added to a compound of formula I, and injections for subcutaneous, intramuscular or intravenous administration can be prepared in the conventional manner.

For the formulation of suppositories, a base, and if desired, surfactants are added to a compound of formula I, and the suppositories are prepared in a conventional manner.

For the formulation of solid preparations for oral administration, an excipient, and if desired, a binder, disintegrator, lubricant, coloring agent, corrigent, flavor etc. are added to a compound of formula I, and then tablets, coated tablets, granules, powders, capsules or the like are prepared in a conventional manner.

The excipients generally used in the art for solid preparations are lactose, sucrose, sodium chloride, starches, calcium carbonate, kaolin, crystalline cellulose, methyl cellulose, glycerin, sodium alginate, gum arabic and the like. The binders generally used in the art for solid preparations are polyvinyl alcohol, polyvinyl ether, polyvinyl pyrrolidone, ethyl cellulose, gum arabic, schellac, sucrose, water, ethanol, propanol, carboxymethyl cellulose, potassium phosphate and the like. The lubricants generally used in the art for solid preparations are magnesium stearate, talc and the like, and further, if desired, include additives such as usual known coloring agents, disintegrators and the like. Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of an aqueous or oleaginous suspension, solution, syrup, elixir and the like, which can be prepared by any conventional way using additives.

The amount of the compound of formula I of the invention to be incorporated into the pharmaceutical composition of the invention varies with the dosage form, solubility and chemical properties of the compound, administration route, administration scheme and the like. Preferably the amount is about 1 to 50 w/w % in the case of oral preparations, and about 0.1 to 5 w/w % in the case of injections which are parenteral preparations.

The dosage of the compound of formula I of the invention is suitably determined depending on the individual cases taking symptoms, age and sex of the subject and the disease severity into consideration. Usually the dosage for oral administration is about 1 to 3 g per day for an adult in 2 to 4 divided doses. The dosage for injection, for example by intravenous administration, is 2 ml (about 1 to 300 mg), which is administered once a day for adults wherein the injection formulation may be diluted with physiological saline or a glucose injection liquid if so desired, and slowly administered over at least 5 minutes. The dosage in case of suppositories is about 1 to 1000 mg, which is administered once or twice a day at an interval of 6 to 12 hours wherein the suppositories are administered by insertion into the rectum

### Example 1

A small piece of fungi from the culture tube was transferred to a malt agar (MA) plate (malt extract 20 g, agar 20 g, deionized water 1L) and allowed to grow at 25° C. for 72 hours. Three pieces of this MA medium containing fungal lawn were transferred to a flask containing 100 ml of UCI media (glucose 25 g, pharamedia 25 g, deionized water 1L). Triplicate were prepared. These flasks were placed on a shaker at 200 rpm, 25° C. for 72 hours. 50 ml of aliquots of this UCI culture were transferred to six separate flasks each containing 1L of UCII media (molasses 20 g, dextrin white 30 g, fish meal 15 g, pharmamedia 15 g, deionized water 1L). These flasks were shaken at 200 rpm for 7 days in an environment maintained at 25° C. The cells were harvested and filtration was performed to separate the mycelia from filtrate. The filtrate was extracted with ethyl acetate (x3) and combined ethyl extract was concentrated. The residue was triturated with methanol. The methanol solution was removed, concentrated and further used for bioassay guided fractionation.

### Example 2

A flash chromatography column was packed with 40u C18 powder. The ratio of gel to sample was 37:1 (w/w). The column was washed with dichloromethane, methanol and then equilibrated with 655 methanol/water. The extract of example 1 was absorbed on clean C₁₈ powder and then applied to the top of column. The following solvent systems were employed (150 ml each) and 18 ml/tube fraction size was collected.

| Fraction no | Solvent |
|---|---|
| 1-8 | 65% methanol/water |
| 9-16 | 75% methanol/water |
| 17-24 | 85% methanol/water |
| 25-32 | 100% methanol |
| 33-40 | 80% dichloromethan/methanol |

Each fraction was analyzed by TLC and similar ones were combined, evaporated to dryness. The combined fraction fractions were analyzed by TLC, NMR and tested for microbial activities.

The TLC conditions used for combining the fraction were as follows:

C18 TLC plate (5 cm height); and

5% water/acetonitrile.

After a TLC run, the chromatography plate was examined visually under a UV lamp, and then sprayed with anisaldehyde and heated. A dark color spot appeared with R_{f} of approximately 0.5.

The combined fractions from primary separation containing Microsphacropsin B and exhibiting microbial activities were subjected to size exclusion chromatography employing a LH-20 column. The day before the column was to be run the gel is swelled in the eluting solvent methanol. The next day the gel was packed in a long, narrow column (1.4 cm id×86 cm l). The ratio of gel to sample was 3:1 (v/wt). The sample was dissolved in methanol and introduced as a solution. A reservoir containing methanol (100 ml) was attached to the top of the column and a fraction collector employed to collect fractions, each consisting of approximately 2 ml. Fractions were examined by TLC, with similar ones combined and concentrated. The pure Microsphaeropsin B was analyzed by UV spectrum, proton NMR, ¹³C NMR, Mass spectrometry and elemental analysis.

Structural Elucidation of Microsphaeropsin B

| ElMS | m/z 399.24 |
|---|---|
| Molecular Formula | C₂₄H₃₃NO₄ |
| Degree of unsaturation | 9 |

1H, ¹³C-NMR, DEPT, HMQC Experiment:

| | | | | | |
|---|---|---|---|---|---|
| 4 | CH3, | 3 | CH2, | 6 | CH, |
| 6 | ═CH, | 1 | ═CH—OH, | 2 | C, |
| 2 | C═O, | | | | |

2-D COSY, HMBC and TOCSY Experiments and its conversion to dehydromicrosphaeropsin and hexahydromicrosphaeropsin confirmed the following structure of Microsphaeropsin B (Formula IV).

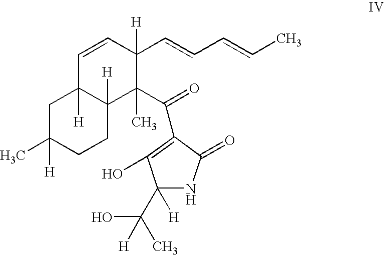

### EXAMPLE 3

### Test for Antibacterial Activities

The compounds of the present invention were tested for minimum inhibitory concentrations (MICs) against the bacteria listed in Table-1 according to the standard microbroth dilution method as described in a NCCLS document. The minimum inhibitory concentrations wer expressed in μg/ml. The test bacteria of 5×10⁵ CFU/ml each were subjected to serial dilutions of the test material under aerobic conditions at 35° C. for 18 hours using Mueller Hinton broth. After incubation, the first concentration of wells to show a complete growth inhibition was considered as the MIC.

TABLE 1
Antibacterial activities (MIC) of the compound of formula II

| | Microsphacropsin B | Vancomycin | Ampicillin |
|---|---|---|---|
| Organism | μg/ml | μg/ml | μg/ml |
| S. aureus ATCC 29213 | 0.5 | 0.5 | 0.062 |
| S. aureus TN1152MRSA | 0.5 | 0.25 8.0 | |
| S. epdermidis | 1.0 | 0.5 | 16.0 |
| ATCC1228 | | | |
| E. faecalis 2353 VSE | 1.0 | 0.25 | 0.25 |
| E. faecalis 2322 VRE | 2.0 | 16.0 | 32.0 |
| E. coli ATCC 25922 | >32 | >32 | 2.0 |

Although some aspects of the present invention were illustrated with the above working examples, the scope of the present invention is not limited to the working examples presented above. Changes in form and detail of the working examples can be made by one skilled in the art based on the recitations of the claims below. The present invention must be measured by the claims and not by the description of the working examples or the preferred embodiments.

What is claimed is:

1. A substantially purified compound of formula I, 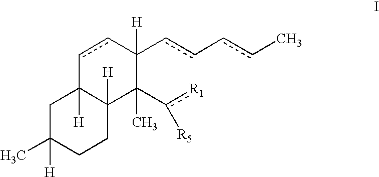
wherein
R₁ is O or OH;
R₅ is a group represented by formula II or III, 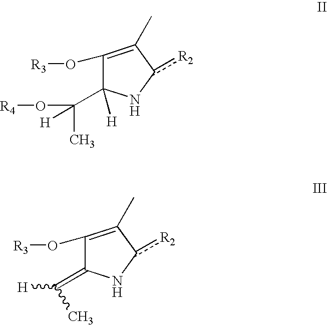
R₂ is O or OH;
each of the dashed lines independently represents a single bond or nothing so that the two atoms attached to the two ends of the dashed line are connected by a single or double bond, wherein the dashed line attached to R₁ represents a single bond when R₁ is O or nothing when R₁ is OH, and the dashed line attached to R₂ represents a single bond when R₂ is O or nothing when R₂ is OH;
R₃ is H, C₁-C₆ alkyl, —C(O)R₁₁, —C(O)R₁₂, —P(O)(OH)₂, an amino acid residue, a dicarboxylic acid residue, —C(O)—C(O)R₁₃ or —C(O)OR₁₄;
R₄ is H, C₁-C₆ alkyl, —C(O)R₄₁, —C(O)R₄₂, —P(O)(OH)₂, an amino acid residue, a dicarboxylic acid residue, —C(O)—C(O)R₄₃ or —C(O)OR₄₄;
the amino acid residue is an amino acid having OH removed from a carboxyl group;
the dicarboxylic acid residue is a dicarboxylic acid having OH removed from one of the carboxyl groups;
R₁ and R₄₁ are each independently (C₁-C₂₀)alkyl, (C₁-C₂₀)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₂₀)alkenyl, (C₂-C₂₀)alkenyl substituted by one to three halogen atoms, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₁₂ and R₄₂ are each independently —N(R₂₀)(R₂₁), 1-imidazolyl or pyrrolyl;
R₂₀ and R₂₁ are each independently H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₁₃ and R₄₃ are each independently (C₁-C₆)alkyl or (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups; and
R₁₄ and R₄₄ are each independently (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
wherein the heterocyclic group is a 5- or 6-membered ring containing one to three N, O and/or S ring atoms optionally fused with a benzene or 5- or 6-membered ring containing one to three N, O and/or S ring atoms; or
a pharmaceutically acceptable salt, solvate or hydrate thereof.

2. The compound of claim 1, wherein
the amino acid residue is —C(O)—C(R₂₄)(R₂₅)—N(R₂₂)(R₂₃), —C(O)—C(R₃₀)(R₃₁)—C(R₂₈)(R₂₉)—N(R₂₆)(R₂₇), —C(O)—C(R₃₈)(R₃₉)—C(R₃₆)(R₃₇)—C(R₃₄)(R₃₅)—N(R₃₂)(R₃₃), 2-pyrrolidinylcarbonyl or 4-hydroxy-2-pyrrolidinylcarbonyl;
R₂₂, R₂₃, R₂₅, R₂₆, R₂₇, R₂₉, R₃₁, R₃₂, R₃₃, R₃₅, R₃₇ and R₃₉ are each independently H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a C₄-C₈ cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₂₄ is H, unsubstituted (C₁-C₆)alkyl or (C₁-C₆)alkyl substituted by one to three substituents selected from thio, OH, (C₁-C₆)alkylthio, (C₁-C₆)alkoxy, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;
one of R₂₈ and R₃₀ is H, (C₁-C₆)alkyl or (C₁-C₆)alkyl substituted by one to three substituents selected from thio, OH, (C₁-C₆)alkylthio, (C₁-C₆)alkoxy, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;
the remaining of R₂₈ and R₃₀ is H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, C₁-C₆ alkyl or C₂-C₆ alkenyl groups, or a cyclic moiety which is a (C₄-C₈)cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
one of R₃₄, R₃₆ and R₃₈ is H, unsubstituted (C₁-C₆)alkyl or (C₁-C₆)alkyl substituted by one to three substituents selected from thio, OH, (C₁-C₆)alkylthio, (C₁-C₆)alkoxy, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;
the remaining of R₃₄, R₃₆ and R₃₈ are H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
wherein the heteroaryl is an aromatic 5- or 6-membered ring containing one to three N, S, and/or O ring atoms optionally fused with a benzene or aromatic 5- or 6-membered ring containing one to three N, S and/or O ring atoms;
a pharmaceutically acceptable salt, solvate or hydrate thereof.

3. The compound of claim 2, wherein
R₂₄ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl;
one of R₂₈ and R₃₀ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl; and
one of R₃₄, R₃₆ and R₃₈ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl; or
a pharmaceutically acceptable salt, solvate or hydrate thereof.

4. The compound of claim 3, wherein
R₅ is the group of formula II;
R₃ and R₄ are each independently H, methyl, ethyl or the amino acid residue; and
at least one of R₃ and R₄ is the amino acid residue; or
a pharmaceutically acceptable salt, solvate or hydrate thereof.

5. The compound of claim 4, wherein
one of R₃ and R₄ is the amino acid residue;
the remaining one of R₃ and R₄ is H;
R₁ and R₂ are O;
each of the dashed lines represents a single bond; or
a pharmaceutically acceptable salt, solvate or hydrate thereof.

6. The compound of claim 5, wherein
R₃ is the amino acid residue; and
R₄ is H; or
a pharmaceutically acceptable salt, solvate or hydrate thereof.

7. The compound of claim 5, wherein
R₄ is the amino acid residue; and
R₃ is H; or
a pharmaceutically acceptable salt, solvate or hydrate thereof.

8. The compound of claim 1, wherein
the dicarboxylic acid residue is —C(O)—R₄₀—C(O)OH;
R₄₀ is a divalent (C₁-C₂₀)hydrocarbyl group, which is unsubstituted or substituted by one to three halogen, OH, SH, nitro, amino, mono-(C₁-C₆)alkyl-amino, di-(C₁-C₆)alkyl-amino or (C₁-C₆)alkyl-carbonyl groups; or
a pharmaceutically acceptable salt, solvate or hydrate thereof.

9. The compound of claim 8, wherein
R₄₀ is (C₁-C₆)alkylene, (C₁-C₆)alkylene substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenylene, (C₂-C₆)alkenylene substituted by one to three halogen atoms, OH, SH, nitro, amino, mono-(C₁-C₆)alkyl-amino, di-(C₁-C₆)alkyl-amino or (C₁-C₆)alkyl-carbonyl groups, a bivalent cyclic moiety which is (C₄-C₈)cycloalkylene, phenylene or naphthylene, —CH₂—R₄₅— or —R₄₅—CH₂—, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₄₅ is 1,2-phenylene, 1,2-phenylene substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, 1,3-phenylene, 1,3-phenylene substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, 1,4-phenylene, or 1,4-phenylene substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups; or
a pharmaceutically acceptable salt, solvate or hydrate thereof.

10. The compound of claim 9, wherein
R₄₀ is —(CH₂)ₙ—, —CH═CH—, —C(CH₃)═CH—, —CH═C(CH₃)—, —C(CH₃)═C(CH₃)—, —CH═CHCH₂—, —C(CH₃)═CHCH₂—, —CH═C(CH₃)CH₂—, —C(CH₃)═C(CH₃)CH₂—, —CH₂CH═CH—, —CH₂C(CH₃)═CH—, —CH₂CH═C(CH₃)—, —CH₂C(CH₃)═C(CH₃)—, —CH₂CH═CHCH₂—, —CH₂C(CH₃)═CHCH₂—, —CH₂CH═C(CH3)CH2—, —CH₂C(CH₃)═C(CH3)CH2—, —C(CH₃)CH═CHCH₂—, —CH₂CH═CHCH(CH₃)—, —CH(CH₃)CH═CHCH(CH₃)—, 1,2-phenylene, 1,3-phenylene or 1,4-phenylene;
n is an integer of 1, 2, 3 or 4; or
a pharmaceutically acceptable salt, solvate or hydrate thereof.

11. The compound of claim 10, wherein
R₅ is the group of formula II;
R₃ and R₄ are each independently H, methyl, ethyl or the dicarboxylic acid residue; and
at least one of R₃ and R₄ is the dicarboxylic acid residue; or
a pharmaceutically acceptable salt, solvate or hydrate thereof.

12. The compound of claim 11, wherein
one of R₃ and R₄ is the dicarboxylic acid residue;
the remaining one of R₃ and R₄ is H;
R₁ and R₂ are O; and
each of the dashed lines represent a single bond; or
a pharmaceutically acceptable salt, solvate or hydrate thereof.

13. The compound of claim 12, wherein
R₃ is the dicarboxylic acid residue;
R₄ is H; or
a pharmaceutically acceptable salt, solvate or hydrate thereof.

14. The compound of claim 12, wherein
R₄ is the dicarboxylic acid residue;
R₃ is H; or
a pharmaceutically acceptable salt, solvate or hydrate thereof.

15. The compound of claim 1, wherein
R₅ is the group of formula II;
R₃ is H, methyl, ethyl or —C(O)—R₁₂;
R₄ is H, methyl, ethyl or —C(O)—R₄₂;
at least R₃ is —C(O)—R₁₂ or R₄ is —C(O)—R₄₂; or
a pharmaceutically acceptable salt, solvate or hydrate thereof.

16. The compound of claim 15, wherein
either R₃ is —C(O)—R₁₂ and R₄ is H, or R₄ is —C(O)—R₄₂ and R₃ is H;
R₁ and R₂ are O; and
each of the dashed lines represents a single bond; or
a pharmaceutically acceptable salt, solvate or hydrate thereof.

17. The compound of claim 16, wherein
R₃ is —C(O)—R₁₂;
R₄ is H; or
a pharmaceutically acceptable salt, solvate or hydrate thereof.

18. The compound of claim 16, wherein
R₄ is —C(O)—R₄₂;
R₃ is H; or
a pharmaceutically acceptable salt, solvate or hydrate thereof.

19. The compound of claim 1, wherein
R₅ is the group of formula II;
R₃ is H, methyl, ethyl or —C(O)—R₁₁;
R₄ is H, methyl, ethyl or —C(O)—R₄₁;
at least R₃ is —C(O)—R₁₁ or R₄ is —C(O)—R₄₁; or
a pharmaceutically acceptable salt, solvate or hydrate thereof.

20. The compound of claim 19, wherein
R₃ is —C(O)—R₁₁ or R₄ is —C(O)—R₄₁;
the remaining one of R₃ and R₄ is H;
R₁ and R₂ are O; and
each of the dashed lines represent a single bond; or
a pharmaceutically acceptable salt, solvate or hydrate thereof.

21. The compound of claim 20, wherein
R₃ is —C(O)—R₁₁;
R₄ is H; or
a pharmaceutically acceptable salt, solvate or hydrate thereof.

22. The compound of claim 20, wherein
R₄ is —C(O)—R₄₁;
R₃ is H; or
a pharmaceutically acceptable salt, solvate or hydrate thereof.

23. The compound of claim 1, wherein
R₅ is the group of formula II;
R₃ and R₄ are each independently H, methyl, ethyl or —P(O)(OH)₂; and
at least one of R₃ and R₄ is —P(O)(OH)₂; or
a pharmaceutically acceptable salt, solvate or hydrate thereof.

24. The compound of claim 23, wherein
one of R₃ and R₄ is —P(O)(OH)₂;
the remaining one of R₃ and R₄ is H;
R₁ and R₂ are O; and
each of the dashed lines represent a single bond; or
a pharmaceutically acceptable salt, solvate or hydrate thereof.

25. The compound of claim 24, wherein
R₃ is —P(O)(OH)₂;
R₄ is H; or
a pharmaceutically acceptable salt, solvate or hydrate thereof.

26. The compound of claim 24, wherein
R₄ is —P(O)(OH)₂;
R₃ is H; or
a pharmaceutically acceptable salt, solvate or hydrate thereof.

27. The compound of claim 1, wherein
R₅ is the group of formula II;
R₃ is H, methyl, ethyl or —C(O)—C(O)—R₁₃;
R₄ is H, methyl, ethyl or —C(O)—C(O)—R₄₃;
at least one R₃ is —C(O)—C(O)—R₁₃ or R₄ is —C(O)—C(O)—R₄₃;
R₁ and R₂ are O; and
each of the dashed lines represents a single bond; or
a pharmaceutically acceptable salt, solvate or hydrate thereof.

28. The compound of claim 1, wherein
R₅ is the group of formula II;
R₃ is H, methyl, ethyl or —C(O)—OR₁₄;
R₄ is H, methyl, ethyl or —C(O)—O—R₄₄;
at least R₃ is —C(O)—OR₁₄ or R₄ is —C(O)—O—R₄₄;
R₁ and R₂ are O; and
each of the dashed lines represents a single bond; or
a pharmaceutically acceptable salt, solvate or hydrate thereof.

29. A compound according to claim 1 represented by formula IV, 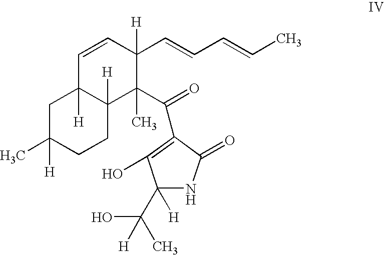
or a pharmaceutically acceptable salt, solvate or hydrate thereof.

30. A pharmaceutical composition suitable for treating a bacterial disease, comprising the compound of claim 1 and a pharmaceutically acceptable carrier or excipient.

31. A pharmaceutical composition suitable for treating a bacterial disease, comprising the compound of claim 29 and a pharmaceutically acceptable carrier or excipient.

32. A method for the treatment or prevention of a disease caused by a bacteria in a subject in need of the treatment or prevention, comprising administering an anti-bacterial effective amount of the compound of claim 1 to said subject.

33. The method of claim 32, wherein the bacteria is a gram-positive pathogen.

34. The method of claim 33, wherein the gram-positive pathogen is Vancomycin resistant Enterococcus faecalis.

35. A method for the treatment or prevention of a disease caused by a bacteria in a subject in need of the treatment or prevention, comprising administering an anti-bacterial effective amount of the compound of claim 29 to said subject.

36. The method of claim 35, wherein the bacteria is a gram-positive pathogen.

37. The method of claim 36, wherein the gram-positive pathogen is Vancomycin resistant Enterococcus faecalis.

38. The method of claim 32, wherein the compound is administered to said subject in an amount of from 1 mg to 3 g per day.

39. The method of claim 35, wherein the compound is administered to said subject in an amount of from 1 mg to 3 g per day.

40. A process for the production of the compound of claim 29, comprising
cultivating a Microsphaeropsin producing strain of Microsphaeropsis olivacea in a growth promoting medium under aerobic condition at a temperature ranging from 20-30° C. to produce Microsphaeropsin B in the growth medium; and thereafter
isolating Microsphaeropsin B from the growth medium to obtain the compound of claim 29.

41. A compound of formula I, 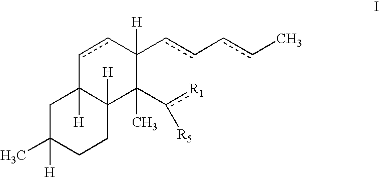
wherein
R₁ is O or OH;
R₅ is a group represented by formula II or III, 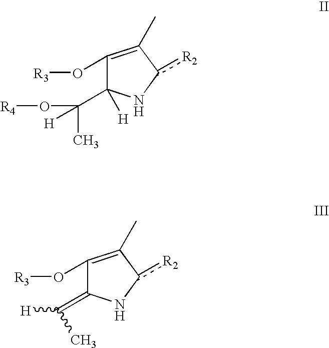
R₂ is O or OH;
each of the dashed lines independently represents a single bond or nothing so that the two atoms attached to the two ends of the dashed line are connected by a single or double bond, wherein the dashed line attached to R₁ represents a single bond when R₁ is O or nothing when R₁ is OH, and the dashed line attached to R₂ represents a single bond when R₂ is O or nothing when R₂is OH;
R₃ is H, C₁-C₆ alkyl, —C(O)R₁₁, —C(O)R₁₂, —P(O)(OH)₂, an amino acid residue, a dicarboxylic acid residue, —C(O)—C(O)R₁₃ or —C(O)OR₁₄;
R₄ is H, C₁-C₆ alkyl, —C(O)R₄₁, —C(O)R₄₂, —P(O)(OH)₂, an amino acid residue, a dicarboxylic acid residue, —C(O)—C(O)R₄₃ or —C(O)OR₄₄;
the amino acid residue is an amino acid having OH removed from a carboxyl group;
the dicarboxylic acid residue is a dicarboxylic acid having OH removed from one of the carboxyl groups;
R₁₂ and R₄₁ are each independently (C₁-C₂₀)alkyl, (C₁-C₂₀)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₂₀)alkenyl, (C₂-C₂₀)alkenyl substituted by one to three halogen atoms, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₁₂ and R₄₂ are each independently —N(R₂₀)(R₂₁), 1-imidazolyl or pyrrolyl;
R₂₀ and R₂₁ are each independently H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₁₃ and R₄₃ are each independently (C₁-C₆)alkyl or (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups; and
R₁₄ and R₄₄ are each independently (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen atoms, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl, phenyl, naphthyl or heterocyclic group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
wherein the heterocyclic group is a 5- or 6-membered ring containing one to three N, O and/or S ring atoms optionally fused with a benzene or 5- or 6-membered ring containing one to three N, O and/or S ring atoms;
with the proviso that a compound of formula IV 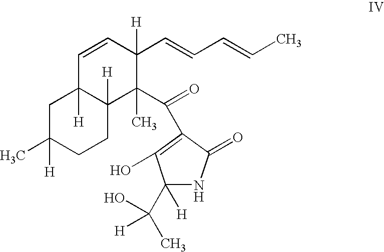
is excluded; or
a pharmaceutically acceptable salt, solvate or hydrate thereof.

42. The compound of claim 41, wherein
the amino acid residue is —C(O)—C(R₂₄)(R₂₅)—N(R₂₂)(R₂₃), —C(O)—C(R₃₀)(R₃₁)—C(R₂₈)(R₂₉)—N(R₂₆)(R₂₇), —C(O)—C(R₃₈)(R₃₉)—C(R₃₆)(R₃₇)—C(R₃₄)(R₃₅)—N(R₃₂)(R₃₃), 2-pyrrolidinylcarbonyl or 4-hydroxy-2-pyrrolidinylcarbonyl;
R₂₂, R₂₃, R₂₅, R₂₆, R₂₇, R₂₉, R₃₁, R₃₂, R₃₃, R₃₅, R₃₇ and R₃₉ are each independently H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a C₄-C₈ cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₂₄ is H, unsubstituted (C₁-C₆)alkyl or (C₁-C₆)alkyl substituted by one to three substituents selected from thio, OH, alkylthio, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;
one of R₂₈ and R₃₀ is H, unsubstituted (C₁-C₆)alkyl or (C₁-C₆)alkyl substituted by one to three substituents selected from thio, OH, alkylthio, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;
the remaining of R₂₈ and R₃₀ is H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, C₁-C₆ alkyl or C₂-C₆ alkenyl groups, or a cyclic moiety which is a (C₄-C₈)cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
one of R₃₄, R₃₆ and R₃₈ is H, unsubstituted (C₁-C₆)alkyl or (C₁-C₆)alkyl substituted by one to three substituents selected from thio, OH, alkylthio, phenyl, hydroxyphenyl, dihydroxyphenyl, trihydroxyphenyl, monohalogenated phenyl, dihalogenated phenyl, trihalogenated phenyl, monoalkylated phenyl, dialkylated phenyl, trialkylated phenyl, heteroaryl, aminocarbonyl, amino, mono-(C₁-C₆)alkyl-amino, di-(C₁-C₆)alkyl-amino, acetylamino, guanidino, (C₁-C₆)alkyl-carbonyl, carboxyl or —OP(O)(OH)₂;
the remaining of R₃₄, R₃₆ and R₃₈ are H, (C₁-C₆)alkyl, (C₁-C₆)alkyl substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenyl, (C₂-C₆)alkenyl substituted by one to three halogen, OH, SH, nitro, amino, mono-(C₁-C₆)alkyl-amino, di-(C₁-C₆)alkyl-amino or (C₁-C₆)alkyl-carbonyl groups, benzyl, benzyl substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, or a cyclic moiety which is a (C₄-C₈)cycloalkyl or phenyl group, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
wherein the heteroaryl is an aromatic 5- or 6-membered ring containing one to three N, S, and/or O ring atoms optionally fused with a benzene or aromatic 5- or 6-membered ring containing one to three N, S and/or O ring atoms;
a pharmaceutically acceptable salt, solvate or hydrate thereof.

43. The compound of claim 42, wherein
R₂₄ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl;
one of R₂₈ and R₃₀ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl; and
one of R₃₄, R₃₆ and R₃₈ is H, methyl, isopropyl, isobutyl, 2-butyl, 2-methylthioethyl, benzyl, 3-indolylmethyl, hydroxymethyl, 1-hydroxyethyl, aminocarbonylmethyl, 2-aminocarbonylethyl, 4-hydroxybenzyl, thiomethyl, 4-aminobutyl, 3-guanidinopropyl, 5-imidazolylmethyl, carboxylmethyl, 2-carboxylethyl, 4-(N,N,N-trimethylamino)butyl, 4-amino-3-hydroxybutyl, —CH₂—OP(O)(OH)₂, 2,2-dicarboxylethyl, 4-(acetylamino)butyl or 3-aminopropyl; or
a pharmaceutically acceptable salt, solvate or hydrate thereof.

44. The compound of claim 43, wherein
R₅ is the group of formula II;
R₃ and R₄ are each independently H, methyl, ethyl or the amino acid residue; and
at least one of R₃ and R₄ is the amino acid residue; or
a pharmaceutically acceptable salt, solvate or hydrate thereof.

45. The compound of claim 44, wherein
one of R₃ and R₄ is the amino acid residue;
the remaining one of R₃ and R₄ is H;
R₁ and R₂ are O;
each of the dashed lines represents a single bond; or
a pharmaceutically acceptable salt, solvate or hydrate thereof.

46. The compound of claim 45, wherein
R₃ is the amino acid residue; and
R₄ is H; or
a pharmaceutically acceptable salt, solvate or hydrate thereof.

47. The compound of claim 45, wherein
R₄ is the amino acid residue; and
R₃ is H; or
a pharmaceutically acceptable salt, solvate or hydrate thereof.

48. The compound of claim 41, wherein
the dicarboxylic acid residue is —C(O)—R₄₀—C(O)OH;
R₄₀ is a divalent (C₁-C₂₀)hydrocarbyl group, which is unsubstituted or substituted by one to three halogen, OH, SH, nitro, amino, mono-(C₁-C₆)alkyl-amino, di-(C₁-C₆)alkyl-amino or (C₁-C₆)alkyl-carbonyl groups; or
a pharmaceutically acceptable salt, solvate or hydrate thereof.

49. The compound of claim 48, wherein
R₄₀ is (C₁-C₆)alkylene, (C₁-C₆)alkylene substituted by one to three halogen, OH, SH, nitro, amino, mono-(C₁-C₆)alkyl-amino, di-(C₁-C₆)alkyl-amino or (C₁-C₆)alkyl-carbonyl groups, (C₂-C₆)alkenylene, (C₂-C₆)alkenylene substituted by one to three halogen atoms, OH, SH, nitro, amino, mono-(C₁-C₆)alkyl-amino, di-(C₁-C₆)alkyl-amino or (C₁-C₆)alkyl-carbonyl groups, a bivalent cyclic moiety which is (C₄-C₈)cycloalkylene, phenylene or naphthylene, —CH₂—R₄₅— or —R₄₅—CH₂—, wherein the cyclic moiety is unsubstituted or substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups;
R₄₅ is 1,2-phenylene, 1,2-phenylene substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, 1,3-phenylene, 1,3-phenylene substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups, 1,4-phenylene, or 1,4-phenylene substituted by one to three halogen, OH, SH, nitro, amino, mono-(C₁-C₆)alkyl-amino, di-(C₁-C₆)alkyl-amino, (C₁-C₆)alkyl-carbonyl, (C₁-C₆)alkyl or (C₂-C₆)alkenyl groups; or
a pharmaceutically acceptable salt, solvate or hydrate thereof.

50. The compound of claim 49, wherein
R₄₀ is —(CH₂)ₙ—, —CH═CH—, —C(CH₃)═CH—, —CH═C(CH₃)—, —C(CH₃)═C(CH₃)—, —CH═CHCH₂—, —C(CH₃)═CHCH₂—, —CH═C(CH₃)CH₂—, —C(CH₃)═C(CH₃)CH₂—, —CH₂CH═CH—, —CH₂C(CH₃)═CH—, —CH₂CH═C(CH₃)—, —CH₂C(CH₃)═C(CH₃)—, —CH₂CH═CHCH₂—, —CH₂C(CH₃)═CHCH₂—, —CH₂CH═C(CH3)CH2—, —CH₂C(CH₃)═C(CH3)CH2—, —C(CH₃)CH═CHCH₂—, —CH₂CH═CHCH(CH₃)—, —CH(CH₃)CH=CHCH(CH₃)—, 1,2-phenylene, 1,3-phenylene or 1,4-phenylene;
n is an integer of 1, 2, 3 or 4; or
a pharmaceutically acceptable salt, solvate or hydrate thereof.

51. The compound of claim 50, wherein
R₅ is the group of formula II;
R₃ and R₄ are each independently H, methyl, ethyl or the dicarboxylic acid residue; and
at least one of R₃ and R₄ is the dicarboxylic acid residue; or
a pharmaceutically acceptable salt, solvate or hydrate thereof.

52. The compound of claim 51, wherein
one of R₃ and R₄ is the dicarboxylic acid residue;
the remaining one of R₃ and R₄ is H;
R₁ and R₂ are O; and
each of the dashed lines represent a single bond; or
a pharmaceutically acceptable salt, solvate or hydrate thereof.

53. The compound of claim 52, wherein
R₃ is the dicarboxylic acid residue;
R₄is H; or
a pharmaceutically acceptable salt, solvate or hydrate thereof.

54. The compound of claim 52, wherein
R₄ is the dicarboxylic acid residue;
R₃ is H; or
a pharmaceutically acceptable salt, solvate or hydrate thereof.

55. The compound of claim 41, wherein
R₅ is the group of formula II;
R₃ is H, methyl, ethyl or —C(O)—R₁₂;
R₄ is H, methyl, ethyl or —C(O)—R₄₂;
at least R₃ is —C(O)—R₁₂ or R₄ is —C(O)—R₄₂; or
a pharmaceutically acceptable salt, solvate or hydrate thereof.

56. The compound of claim 55, wherein
either R₃ is —C(O)—R₁₂ or R₄ is —C(O)—R₄₂;
the remaining one of R₃ and R₄ is H;
R₁ and R₂ are O; and
each of the dashed lines represents a single bond; or
a pharmaceutically acceptable salt, solvate or hydrate thereof.

57. The compound of claim 56, wherein
R₃ is —C(O)—R₁₂;
R₄ is H; or
a pharmaceutically acceptable salt, solvate or hydrate thereof.

58. The compound of claim 56, wherein
R₄ is —C(O)—R₄₂;
R₃ is H; or
a pharmaceutically acceptable salt, solvate or hydrate thereof.

59. The compound of claim 41, wherein
R₅ is the group of formula II;
R₃ is H, methyl, ethyl or —C(O)—R₁₁;
R₄ is H, methyl, ethyl or —C(O)—R₄₁;
at least R₃ is —C(O)—R₁₁ or R₄ is —C(O)—R₄₁; or
a pharmaceutically acceptable salt, solvate or hydrate thereof.

60. The compound of claim 59, wherein
either R₃ is —C(O)—R₁₁ or R₄ is —C(O)—R₄₁;
the remaining one of R₃ and R₄ is H;
R₁ and R₂ are O; and
each of the dashed lines represent a single bond; or
a pharmaceutically acceptable salt, solvate or hydrate thereof.

61. The compound of claim 60, wherein
R₃ is —C(O)—R₁₁;
R₄ is H; or
a pharmaceutically acceptable salt, solvate or hydrate thereof.

62. The compound of claim 60, wherein
R₄ is —C(O)—R₄₁;
R₃ is H; or
a pharmaceutically acceptable salt, solvate or hydrate thereof.

63. The compound of claim 41, wherein
R₅ is the group of formula II;
R₃ and R₄ are each independently H, methyl, ethyl or —P(O)(OH)₂; and at least one of R₃ and R₄ is —P(O)(OH)₂; or
a pharmaceutically acceptable salt, solvate or hydrate thereof.

64. The compound of claim 63, wherein
one of R₃ and R₄ is —P(O)(OH)₂;
the remaining one of R₃ and R₄ is H;
R₁ and R₂ are O; and
each of the dashed lines represent a single bond; or
a pharmaceutically acceptable salt, solvate or hydrate thereof.

65. The compound of claim 64, wherein
R₃ is —P(O)(OH)₂;
R₄ is H; or
a pharmaceutically acceptable salt, solvate or hydrate thereof.

66. The compound of claim 64, wherein
R₄ is —P(O)(OH)₂;
R₃ is H; or
a pharmaceutically acceptable salt, solvate or hydrate thereof.

67. The compound of claim 41, wherein
R₅ is the group of formula II;
R₃ is H, methyl, ethyl or —C(O)—C(O)—R₁₃;
R₄ is H, methyl, ethyl or —C(O)—C(O)—R₄₃;
at least R₃ is —C(O)—C(O)—R₁₃ or R₄ is —C(O)—C(O)—R₄₃;
R₁ and R₂ are O; and
each of the dashed lines represents a single bond; or
a pharmaceutically acceptable salt, solvate or hydrate thereof.

68. The compound of claim 41, wherein
R₅ is the group of formula II;
R₃ is H, methyl, ethyl or —C(O)—O—R₁₄;
R₄ is H, methyl, ethyl or —C(O)—O—R₄₄;
at least R₃ is —C(O)—O—R₁₄ or R₄ is —C(O)—O—R₄₄;
R₁ and R₂ are O; and
each of the dashed lines represents a single bond; or
a pharmaceutically acceptable salt, solvate or hydrate thereof.

69. The compound of claim 1, wherein R₅ is a group represented by formula II; or
a pharmaceutically acceptable salt, solvate or hydrate thereof.

70. The compound of claim 69, wherein R₁ and R₂ are O; or
a pharmaceutically acceptable salt, solvate or hydrate thereof.

71. The compound of claim 70, wherein R₃ is H; or
a pharmaceutically acceptable salt, solvate or hydrate thereof.

72. The compound of claim 70, wherein R₄ is H; or
a pharmaceutically acceptable salt, solvate or hydrate thereof.

73. The compound of claim 1, wherein R₅ is a group represented by formula III; or
a pharmaceutically acceptable salt, solvate or hydrate thereof.

74. The compound of claim 73, wherein R₁ and R₂ are O; or
a pharmaceutically acceptable salt, solvate or hydrate thereof.

75. The compound of claim 74, wherein R₃ is H; or
a pharmaceutically acceptable salt, solvate or hydrate thereof.

76. The compound of claim 41, wherein R₅ is a group represented by formula II; or
a pharmaceutically acceptable salt, solvate or hydrate thereof.

77. The compound of claim 76, wherein R₁ and R₂ are O; or
a pharmaceutically acceptable salt, solvate or hydrate thereof.

78. The compound of claim 77, wherein R₃ is H; or
a pharmaceutically acceptable salt, solvate or hydrate thereof.

79. The compound of claim 77, wherein R₄ is H; or
a pharmaceutically acceptable salt, solvate or hydrate thereof.

80. The compound of claim 41, wherein R₅ is a group represented by formula III; or
a pharmaceutically acceptable salt, solvate or hydrate thereof.

81. The compound of claim 80, wherein R₁ and R₂ are O; or
a pharmaceutically acceptable salt, solvate or hydrate thereof.

82. The compound of claim 81, wherein R₃ is H; or
a pharmaceutically acceptable salt, solvate or hydrate thereof.
